(19) United States
(12) Patent Application Publication
(10) Pub. No.: US 2007/0227540 A1
(43) Pub. Date: Oct. 4, 2007

(54) Control Valve for a Ventilator

(75) Inventors: Lars Ljungberg, Floda (SE); Mikael Tiedje, Hisings Backa (SE); Staffan Bengtsson, Goteborg (SE)

Correspondence Address: RAYMOND R. MOSER JR., ESQ.;MOSER IP LAW GROUP, 1040 BROAD STREET, 2ND FLOOR, SHREWSBURY, NJ, 07702, US

(73) Assignee: Breas Medical AB, Molnlycke (SE)

(21) Appl. No.: 10/599,677

(22) PCT Filed: Apr. 5, 2005
(86) PCT No.: PCT/SE05/00493

§ 371 Date: May 14, 2007

Related U.S. Application Data
(60) Provisional application No. 60/573,230, filed on May 21, 2004.

(30) Foreign Application Priority Data
Apr. 5, 2004 (SE) 0400890-0

(51) Int. Cl. A61M 16/20

(52) U.S. Cl. 128/205.24

(57) ABSTRACT

A ventilator for supplying breathable gas to the airway of a patient with a respiratory disorder is provided herein an, in one embodiment, includes a gas flow generator for generating a flow of said breathable gas to the patient, said gas flow generator comprising a gas flow generator chamber provided with a gas inlet opening and a gas outlet opening; a control valve for controlling the flow and/or pressure of the gas distributed to the patient, said control valve comprising a valve body which is rotatably arranged about a rotational axis within a valve chamber. The valve body essentially exhibits the shape of a sector of a circle, in such a way that an arced first flow regulatory surface is formed along the circular arc of said sector, and that second and third essentially straight flow regulatory surfaces, respectively, are formed along the two diverging sides of said sector.

### FIELD OF THE INVENTION

The present invention relates to a ventilator for supplying breathable gas, normally air, at elevated pressure to a patient for treating breathing disorders such as for example Obstructive Sleep Apnea (OSA), Cheyne-Stokes respiration or emphysema. More particularly, the ventilator comprises a novel control valve design, which is simple and cheap to manufacture, and which may effectively be used in a compact space. The ventilator may also be used in the treatment of cardiac disorders, such as Congestive Heart Failure (CHF). The invention is applicable to advanced intensive care ventilators for assisted ventilation or Continuous Positive Airway Pressure ventilators (CPAP). The novel control valve design provides smooth and effective flow regulating characteristics and a reduced overall size of the ventilator, thus improving user comfort for the patients.

### BACKGROUND OF THE INVENTION

Ventilators for supplying breathable gas to the airway of a patient, are well known in the art per se. In the simplest form of CPAP therapy (not applicable in the present invention), air of a constant positive pressure is supplied to the airway of a patient, in order to treat Obstructive Sleep Apnea (OSA). The required pressure level varies for individual patients and their respective breathing disorders. CPAP therapy may be applied not only to the treatment of breathing disorders, but also to the treatment of Congestive Heart Failure (CHF). These simple CPAP devices generally do not include a control valve, but are included herein for reference only.

A more advanced form of CPAP therapy is commonly referred to as Bi-Level CPAP, wherein air is applied to the airway of a patient alternatively at a higher pressure level during inspiration and a lower pressure level during expiration. The higher pressure level is referred to as IPAP (Inspiratory Positive Airway Pressure), whilst the lower pressure level is referred to as EPAP (Expiratory Positive Airway Pressure). In a Bi-level CPAP ventilator, EPAP and IPAP are thus synchronized with the patient's inspiratory cycle and expiratory cycle so that the patient will not be forced to overcome a high pressure from the ventilator during the expiration phase of his or her breathing. Consequently, Bi-Level CPAP ventilators generally provides improved breathing comfort for the patient compared to the simpler “single level” CPAP ventilator described initially. In order to detect the patients transition from the inspiratory breathing phase to the expiratory breathing phase, a Bi-Level CPAP ventilator is provided with one or more sensors. Normally, a flow sensor is located somewhere along the air supply conduit to the patient. Additionally, a pressure sensor may for example be located in a patient interface means, such as a facial mask, or along the air supply conduit. The different pressure levels and/or flow levels are normally controlled by means of a control valve, which restricts and directs the airflow in various ways. As will be described in more detail below, modern ventilators often use a gas flow generator in the form of an electric fan unit, and the pressure and/or flow may thus be additionally or exclusively controlled by varying the rotary speed of the fan.

Another, yet more advanced type of CPAP ventilator is generally referred to as an AutoCPAP ventilator. Other terms for this type of ventilator include: Auto Adaptive CPAP (AACPAP), Auto Titration CPAP or Self-titrating individual AutoCPAP. In this description, these terms will commonly be referred to as an AutoCPAP ventilators for the sake of clarity. Here, IPAP and EPAP as well as other relevant parameters are automatically changed with respect to specific detected breathing patterns significative of different breathing disorders or phases thereof. This is an “intelligent” form of CPAP treatment, in which a certain condition may even be foreseen by the ventilator before the condition is felt by the patient, and wherein a suitable combination of IPAP and EPAP as well as other relevant parameters are applied in order to treat or alleviate the symptoms of the patient. For this purpose, it is known to provide a ventilator with an integral learning artificial neural network (ANN) to gather large amounts of relevant breathing data from a vast population of patients with breathing disorders worldwide. The ANN is able to detect and identify breathing patterns that are symptomatic of a certain condition or disorder and to then automatically adapt the ventilator parameter settings for effecting a relevant treatment pattern at an early stage. Apart from added control hardware, software and more sensors, the basic hardware design of an AutoCPAP ventilator may be substantially identical a Bi-Level CPAP ventilator.

A trend in modern ventilator technology is directed toward ever more compact and lightweight CPAP ventilators, that are unobtrusive at the bedside, offer increased mobility for patients and generally have a less “hospital-like” design, in order to improve user comfort.

A ventilator of the above mentioned type includes a gas flow generator for creating a gas flow to the patient. A patient interface means, in the form of a facial mask or a tracheal tube is provided for introducing the breathable gas into the airway of the patient.

In older ventilators, the gas flow generator often consisted of an air bellows unit, which was sufficiently quiet, but had to be rather large in order to effectively produce the required airflow. Thus, in modern, more compact ventilators, a compact but effective electric fan unit has replaced the air bellows often found in older systems.

In the more the advanced CPAP ventilators, such as the Bi-level CPAP or AACPAP mentioned above, a control valve is provided for controlling the flow and/or pressure of the gas from the gas flow generator. The simplest form of CPAP ventilator lacks this feature, and is thus not covered by the present invention. The control valve comprises a valve body, which is movably arranged within a valve chamber.

However, even in the more modern conventional ventilators, the control valve is traditionally designed and manufactured as a separate assembly within the ventilator and is connected to the gas flow generator by means of an interconnecting pipe or hose conduit section of various lengths depending on the layout of a specific ventilator. Hence, partly for this reason, conventional ventilators tend to be unnecessarily bulky.

### OBJECT OF THE INVENTION

It is the object of the present invention to provide a simple and compact control valve which provides smooth, reliable and effective flow regulating characteristics and a reduced overall size of the ventilator, when compared to currently available ventilators on the market, as well as to reduce the manufacturing cost of the control valve.

### SUMMARY OF THE INVENTION

The above mentioned object is achieved by the invention providing a ventilator for supplying breathable gas to the airway of a patient with a respiratory disorder, comprising:

a gas flow generator, such as an electric fan, for generating a flow of said breathable gas to the patient, said gas flow generator comprising a gas flow generator chamber provided with a gas inlet opening and a gas outlet opening;

a control valve for controlling the flow and/or pressure of the gas distributed to the patient, said control valve comprising a valve body which is rotatably arranged about a rotational axis within a valve chamber. The invention is especially characterized in,

that the rotational axis of the valve body is substantially perpendicular to the exhaust direction of the breathable gas at the gas outlet opening of the gas flow generator;

that the valve body essentially exhibits the shape of a sector of a circle in a plane perpendicular to said rotational axis, in such a way that an arced first flow regulatory surface is formed along the circular arc of said sector, and that second and third essentially straight flow regulatory surfaces, respectively, are formed along the two diverging sides of said sector;

that said valve chamber exhibits two mutually opposing, essentially flat sidewalls both extending in a plane perpendicular to said rotational axis of the valve body, and

that first, second and third valve body abutment surfaces, respectively, extend between said sidewalls of the valve chamber, said valve body abutment surfaces being arranged for abutting contact with the arced first flow regulatory surface of the valve body, depending on the angular position of the valve body within the valve chamber, wherein

said first valve body abutment surface is located on one side of an inlet opening to the valve chamber, said inlet opening being connected to the gas outlet opening of the gas flow generator chamber;

said second valve body abutment surface is located between said inlet opening and a bypass opening arranged for directing a portion of the gas flow back into said gas flow generator via a bypass conduit connected to the gas inlet opening of the gas flow generator chamber, and

said third valve body abutment surface is located on an opposing side of said bypass opening with respect to said second valve body abutment surface.

In an advantageous embodiment of the invention, the valve body exhibits rounded transitional portions between the arced first flow regulatory surface and the second and a third essentially straight flow regulatory surfaces.

In one embodiment, the valve body is formed in such a way that a sector angle between the second and third flow regulatory surfaces is between 90°-160°. However, the sector angle is preferably between 110°-130°, and is most preferably 120°.

In a favorable embodiment of the invention, the gas flow generator chamber and said valve chamber are integrally formed in a combined gas flow generator & control valve housing, and that

said valve chamber is located in immediate conjunction to the gas outlet opening of the gas flow generator chamber within said combined gas flow generator & valve housing.

Preferably, the gas outlet opening of the gas flow generator chamber also defines an inlet opening to the valve chamber.

Further, the rotational axis of the valve body is preferably parallel to a rotational axis of a fan rotor wheel in said gas flow generator chamber.

In a well functioning embodiment of the present invention, an electric stepper motor is attached to the combined gas flow generator & control valve housing, said electric stepper motor having a stepper motor shaft coupled to the valve body in said valve chamber.

Suitably, the valve body is provided with a through hole, said through hole having a cross-sectional shape such that the valve body is rotationally fixed relative to the stepper motor shaft, whilst being freely slidably arranged in an axial direction of said stepper motor shaft for easy insertion or removal of the valve body in the valve chamber.

Further features and advantages of the invention will be described in the detailed description of embodiments below.

### BRIEF DESCRIPTION OF THE DRAWINGS

The invention will now be described in greater detail by way of example only and with reference to the attached drawings, in which

FIG. 1 shows a schematic view of a ventilator according to the present invention;

FIG. 2 shows a perspective view of an exemplifying embodiment of a combined gas flow generator & control valve housing according to the invention, shown with one shell removed in order to expose the internals of the housing;

FIG. 3 shows an elevational view of a combined gas flow generator & control valve housing according to the invention, shown with one shell removed in order to expose the internals of the housing. The control valve is shown in its fully open position;

FIG. 4 shows a detailed, separate top view of the valve body according to the invention;

FIG. 5 shows a separate perspective view of the valve body according to the invention;

FIG. 6 shows an elevational view of a combined gas flow generator & control valve housing as seen in FIG. 3, but with the control valve in its partly open, flow regulating position;

FIG. 7 shows an elevational view of a combined gas flow generator & control valve housing as seen in FIGS. 3 and 6, but with the control valve in its partly open, flow regulating and bypass position;

FIG. 8 shows an elevational view of a combined gas flow generator & control valve housing as seen in FIGS. 3, 6 and 7 but with the control valve in its closed position;

FIG. 9 shows a schematic view of the valve opening extent, as seen at the inlet opening to the valve chamber, the control valve being in its fully open position as shown in FIG. 3;

FIG. 10 shows a schematic view of the valve opening extent, like in FIG. 9, but with the control valve in its partly open, flow regulating position as shown in FIG. 6, and

FIG. 11 finally shows a schematic view of the valve opening extent, like in FIGS. 9 and 10, but with the control valve in its fully closed position as shown in FIG. 8.

### DESCRIPTION OF EXEMPLIFYING EMBODIMENTS

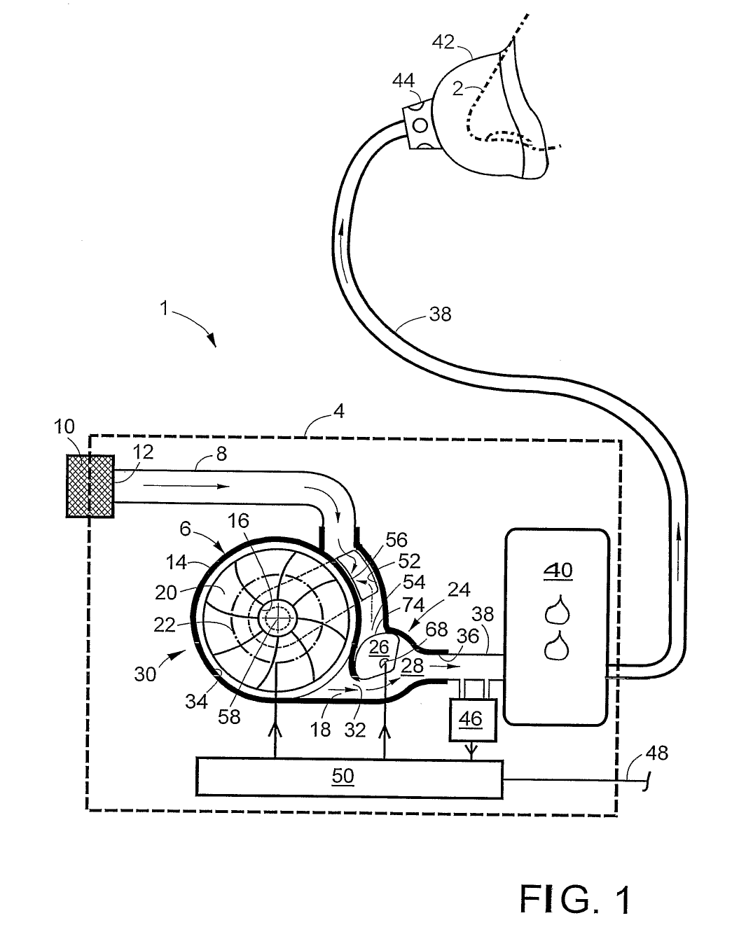

In FIG. 1, reference numeral 1 denotes a ventilator for supplying breathable gas—normally air—into the airway of a patient for treating breathing disorders such as for example Obstructive Sleep Apnea (OSA), Cheyne-Stokes respiration or emphysema. In the figure, a schematically drawn nose 2 of a patient is shown with dash-dotted lines. It should be noted that the schematic FIG. 1 is drawn in a highly simplified way in order to clearly illustrate the basic features of the invention. Thus, a production version of a ventilator according to the invention may look significantly different than in the shown illustrations, although the basic features are still present.

As mentioned in the background above, the ventilator is either of the initially described Bi-Level CPAP type or the AutoCPAP-type.

The ventilator 1 has an external housing 4, schematically illustrated with dashed lines in FIG. 1. A gas flow generator 6 is located within the external housing 4. In the preferred example, the gas flow generator 6 is an electric fan, adapted for generating a flow of breathable gas to the patient. The gas flow generator 6 draws in air (or any other breathable gas) via a gas inlet conduit 8. A particle filter 10 is provided at an external opening 12 of the gas inlet conduit 8 in order to stop undesired particular matter from entering the ventilator 1.

More particularly, the gas flow generator 6 comprises a generally circular gas flow generator chamber 14 provided with a gas inlet opening 16 and a gas outlet opening 18, respectively. As shown in FIG. 1, the gas flow generator 6 further comprises a fan rotor wheel 20 arranged within the gas flow generator chamber 14. The fan rotor wheel 20 is driven by an electric motor 22, which is schematically drawn with dash-dotted lines behind the fan rotor wheel 20. The electric motor 22 is preferably of a known compact type, wherein a stator (not shown) is fixedly attached to the combined gas flow generator & valve housing 30, and a rotor (not shown) is fixedly attached to said fan rotor wheel 20, the latter of course being rotatably journalled in the housing 30.

The ventilator 1 further comprises a control valve 24 for controlling the flow and/or pressure of the gas distributed to the patient. The control valve 24, in turn, comprises a valve body 26, which is movably arranged within a valve chamber 28.

As is clearly shown in FIG. 1, the gas flow generator chamber 14 and the valve chamber 28 are integrally formed in a combined gas flow generator & control valve housing 30, as drawn with bold black lines in the schematical FIG. 1. Moreover, the valve chamber 28 is located in immediate conjunction to the gas outlet opening 18 of the gas flow generator chamber 14 within said combined gas flow generator & valve housing 30. By the term “immediate conjunction” is here meant no intermediate conduit extends between the gas flow generator chamber 14 and the valve chamber 28. Thus, in the shown embodiment, the gas outlet opening 18 of the gas flow generator chamber 14 also defines an inlet opening 32 to said valve chamber 28.

In the embodiment shown in FIG. 1, the gas outlet opening 18 of said gas flow generator chamber 14 and said inlet opening 32 of said valve chamber 28 are formed in a peripheral outer wall 34 of said gas flow generator chamber 14.

As seen in the right end of the combined gas flow generator & control valve housing 30 in FIG. 1, the valve chamber 28 is also provided with an outlet opening 36. The outlet opening 36 is connected to an outlet conduit 38, which via an air humidifier 40 is connected to a patient interface means 42. The air humidifier 40 may be of a type well known per se and will thus not be described further here. The patient interface means 42 is adapted for introducing the breathable gas into the airway of said patient, and here includes a facial mask adapted for non-invasive attachment over the nose 2 of a patient. Exhaust openings 44, or “leakage holes” for venting exhaled air from the patient are provided on the patient interface means 42. The exhaust openings 44 may also include a valve (not shown). Alternatively, the patient interface means 42 instead includes a tracheal tube (not shown) for invasive insertion in the trachea of a patient. The external extension of the outlet conduit 38 is preferably a flexible hose.

In the shown example, a flow sensor 46 is located along the outlet conduit 38. The flow sensor 46, along with other optional sensors (Not shown, but as indicated as a symbolic input line 48) provides input for a control unit 50. The control unit 50 then controls either the speed of the electric motor 22, and thereby the fan rotor wheel 20, or the position of the valve body 26 within the valve chamber 28, or both, in order to provide an appropriate gas flow or pressure to the patient, depending—for example—on if he or she is in an inspiratory phase or an expiratory phase of breathing. Many ways and modes of controlling a Bi-Level CPAP or an AutoCPAP ventilator are known in the art, and will thus not be further described herein. In FIG. 1, the valve body 26 is in a fully open position, wherein full gas flow provided by the gas flow generator 6 is distributed to the patient, as indicated by the arrows.

In some situations, requiring a lesser gas flow to the patient, some air is passed by the control valve 24 and back into the gas flow generator via a bypass conduit 52, in a manner well known per se. However, in the embodiment shown in FIG. 1, the bypass conduit 52 is integrally formed in the combined gas flow generator & control valve housing 30. The bypass conduit 52 extends from a bypass opening 54 in the valve chamber 28 to the gas inlet opening 16 in the gas flow generator chamber 14. In a favorable embodiment, the bypass conduit 52 extends—at least along a section of its length—along the peripheral outer wall 34 of said gas flow generator chamber 14, said peripheral outer wall here also defining a peripheral inner wall 56 for the bypass conduit 52.

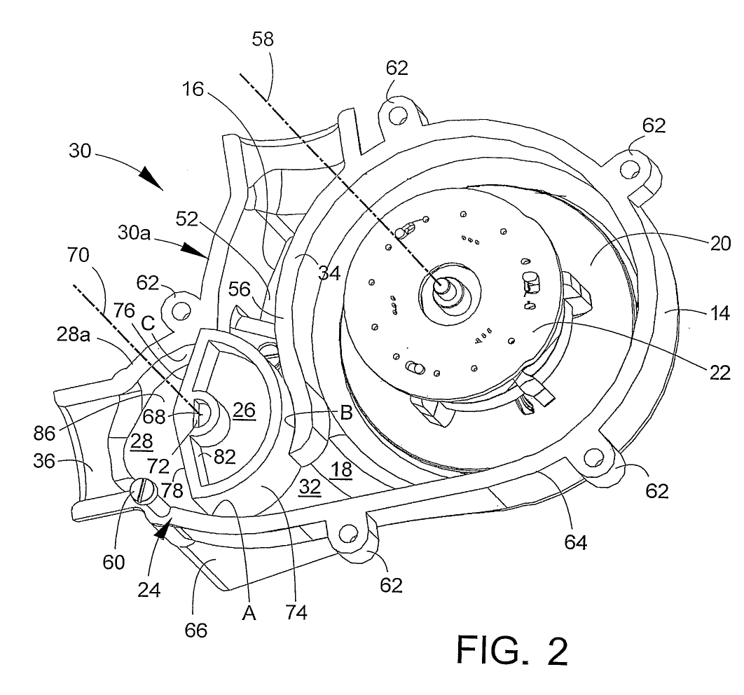

In an embodiment shown in FIG. 2, the combined gas flow generator & control valve housing 30 is structurally divided—in a plane perpendicular to a rotational axis 58 of the fan rotor wheel 20—into a first shell 30a and a second shell 30b (not shown), i.e. the plane of the paper sheet in FIG. 1. FIG. 2 shows the housing 30 with one shell, 30b, removed in order to expose the internals of the housing 30. Hereby, a section 28a, 28b (not shown) of the valve chamber 28 is defined in each of the shells 28a and 28b (not shown). Preferably the two shells 30a, 30b of the combined gas flow generator & control valve housing 30 are made in plastic by means of injection molding. Alternatively, however, the shells 30a, 30b may be made in metal, such as zinc or bronze. In the shown embodiment, the shells 30a and 30b are joined together by means of multiple mounting screws 60 (only one of which is shown) extending through a corresponding number of screw lugs 62 located along the outline periphery 64 of each shell 30a, 30b. A skilled man in the art will of course realize that the shells may alternatively be joined together in other ways, such as for example by means of snap fasteners (not shown).

As is further shown in FIG. 2, an electric stepper motor 66 is attached to the combined gas flow generator & control valve housing 30, said electric stepper motor having a stepper motor shaft 68 coupled to the valve body 26 in the valve chamber 28. The electric stepper motor 66 may alternatively be replaced by another type of motor or turning rotating device adopted to rotate the valve body 26. The valve body 26 is thus rotatably arranged about a rotational axis 70—which coincides with the stepper motor shaft 68 and extends parallelly with the previously mentioned rotational axis 58 of the fan rotor wheel 20. Furthermore, the rotational axis 70 is perpendicular to the exhaust direction of the breathable gas at the gas outlet opening 18 of the gas flow generator 6. In FIG. 2, the rotational axis 70 and the rotational axis 58 are illustrated with dash-dotted lines for the sake of clarity. The valve body 26 is provided with a through hole 72 for the stepper motor shaft 68. The through hole 72 has a cross-sectional shape such that the valve body 26 is rotationally fixed relative to the stepper motor shaft 68, whilst being freely slidably arranged in an axial direction of said stepper motor shaft 68 for easy insertion or removal of the valve body 26 in the valve chamber 28. In the shown example, the cross-sectional shape is semi-circular, but other equally suitable shapes may alternatively be used for the same purpose, such as triangular, rectangular, pentagonal, hexagonal or other polygonal shapes. As illustrated by the semi-circular shape, the cross-sectional shape may also be partially rounded.

As clearly illustrated in the separate view of the valve body in FIG. 4, the valve body 26 according to the present invention essentially exhibits the shape of a sector of a circle in a plane perpendicular to said rotational axis 70. Hence, an arced first flow regulatory surface 74 is formed along the circular arc of the sector, and second and a third essentially straight flow regulatory surfaces, 76 and 78 respectively, are formed along the two diverging sides of the sector-shaped valve body 26. In the exemplifying embodiment shown in FIG. 2, the valve body 26 is formed in such a way that a sector angle α between the second and third flow regulatory surfaces 76, 78 is approximately 120°. In other embodiments of the invention, the sector angle α may be varied within the angular interval 90°-160°, preferably further narrowed to 110°-130°. In comparison, an alternative embodiment of the valve body 26 is shown in FIG. 2, wherein the sector angle α is approximately 140°.

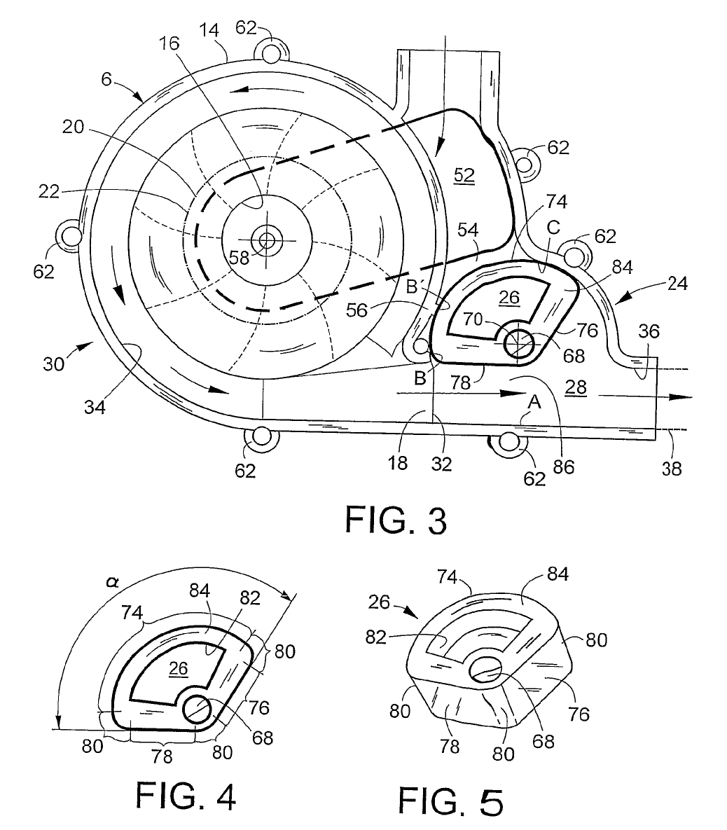

Also, in the embodiment shown in FIGS. 3-8, the valve body 26 exhibits rounded transitional portions 80 between the arced first flow regulatory surface 74 and the second and a third essentially straight flow regulatory surfaces 76 and 78, respectively. It is to be noted, however, that said rounded transitional portions 80 may alternatively be absent, resulting in sharp edges, as shown in the embodiment of FIG. 2. The valve body 26 is preferably molded in plastic, although it may alternatively be made of other materials, including various suitable metals, such as stainless steel, brass or bronze. In the shown embodiments, the valve body 76 is molded with two recesses 82 (one of which is located on the reverse, not shown side of the valve body 26) for manufacturing reasons, i.e. facilitated molding, rather than functional reasons. As clearly shown in the separate perspective view of the valve body 26 in FIG. 5, the recesses 82 are located in two otherwise mutually parallel flat end surfaces 84 of the valve body 26, said flat end surfaces 84 extending perpendicularly to the flow regulatory surfaces 74, 76, 78 mentioned above. The flat end surfaces 84 of the valve body 26 are arranged for a sliding abutment against two mutually opposing, flat sidewalls 86 of the valve chamber 28—one of which sidewalls 86 is visible in FIG. 3 (the other one being located on the removed other half of the valve chamber 28). Both sidewalls 86 extend in a plane perpendicular to said rotational axis 70 of the valve body 26.

The valve chamber 28 exhibits first, second and third valve body abutment surfaces A, B and C, respectively, extending between said sidewalls 86. The valve body abutment surfaces A, B, C are arranged for abutting contact with the arced first flow regulatory surface 74 of the valve body 26, depending on the angular position of the valve body 26 within the valve chamber 26. As shown in FIG. 3, the first valve body abutment surface A is located on one side of an inlet opening 32 to the valve chamber 28, said inlet opening 32 being located at the gas outlet opening 18 of the gas flow generator chamber 14 in this embodiment. The second valve body abutment surface B is located between said inlet opening 32 and a bypass opening 54 arranged for directing a portion of the gas flow back into said gas flow generator 6 via the bypass conduit 52 connected to the gas inlet opening 16 of the gas flow generator chamber 14. In the embodiment shown in FIG. 3, a supplemental second valve body abutment surface B′ is provided next to the second abutment surface B. Such a supplemental second valve body abutment surface B′ is, however, not provided in the embodiment shown in FIG. 2, wherein the second valve body abutment surface B is formed as a continuous surface, rather than the divided one as seen in FIG. 3. The third valve body abutment surface C is located on an opposing side of said bypass opening 54 with respect to said second valve body abutment surface B. With reference now to the series of FIGS. 3, 6-7 and 8, the various operational positions of the control valve 24, and thus the various angular positions of the valve body 26 will now be described in greater detail. Hence, FIG. 3 shows the control valve 24 in its fully open position, in which all available gas flow from the gas flow generator 6 is distributed to the patient during an inspiration phase. In this fully open position, the valve body 26 is oriented in an angular position such that its arced arced, first flow regulatory surface 74 is in abutting contact with the second valve body abutment surface B, the supplemental second valve body abutment surface B′, and the third valve body abutment surface C on the opposing side of the bypass opening 54. Thus, the bypass opening 54 is fully blocked by the valve body 26, allowing no gas flow into the bypass conduit 52. In this position, the straight, third flow regulatory surface 78 on the valve body 26 is substantially parallel with the exhaust flow direction from the gas outlet opening 18 in the gas flow generator chamber 14.

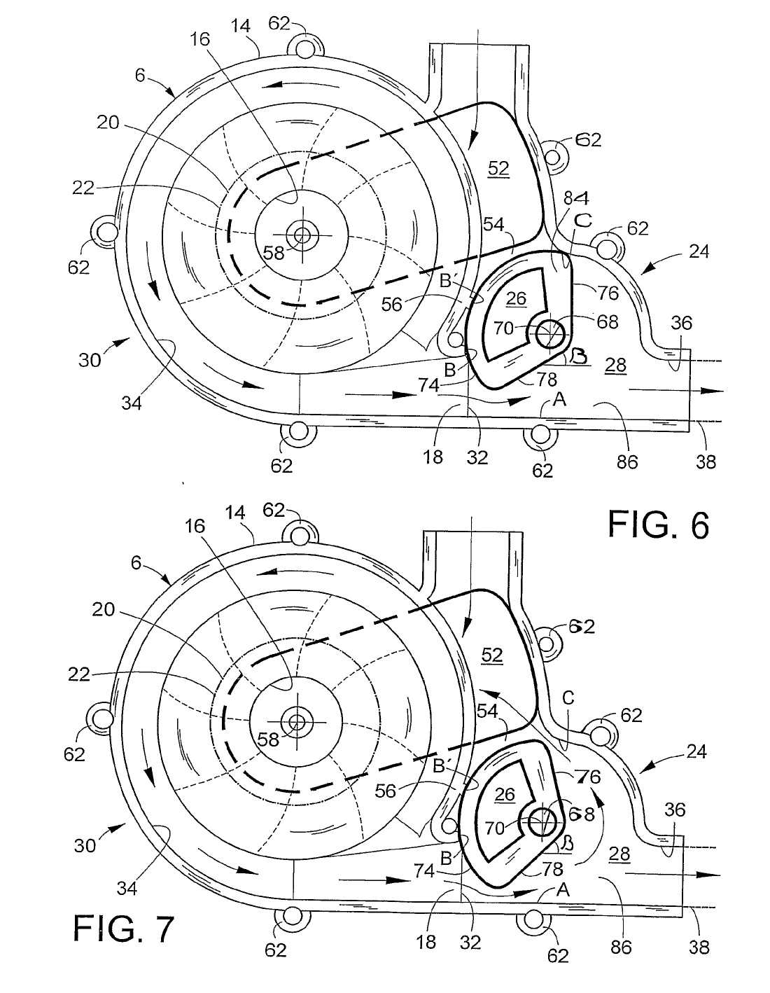

In FIG. 6, the valve body 26 has been rotated counter clockwise with a rotation angle β with respect to the original angular position of the valve body 26 in the fully open position of the control valve 24 (here defined as β=0, as shown in FIG. 3). Hence, in FIG. 6, the control valve 24 is shown in a partly open, flow regulating position. The gas flow to the patient is now restricted to approximately 50%, whilst the bypass opening 54 is still blocked by the valve body 26. The arced, first flow regulatory surface 74 is thus still in abutting contact with the second valve body abutment surface B, the supplemental second valve body abutment surface B′, and the third valve body abutment surface C on the opposing side of the bypass opening 54.

In FIG. 7, the valve body 26 is rotated further in the counter clockwise direction, thus increasing the rotation angle β to such an extent that the arced, first flow regulatory surface 74 of the valve body 26 no longer abuts the third valve body abutment surface C on the opposing side of the bypass opening 54. Now, a bypass gas flow is allowed to pass through the bypass opening 54 and back into the gas flow generator 6 via the bypass conduit 52, in order to utilize the dynamic energy of the gas flow during a regulatory phase. A remaining part of the gas flow is still distributed to the patient. In the shown embodiment, the bypass opening 54 is unblocked by the valve body 26 first since the valve body has reached an angular position corresponding to a flow restriction of approximately 50%. A skilled person will, however, realize that this relationship may be altered within the scope of the invention as defined in the appended claim 1.

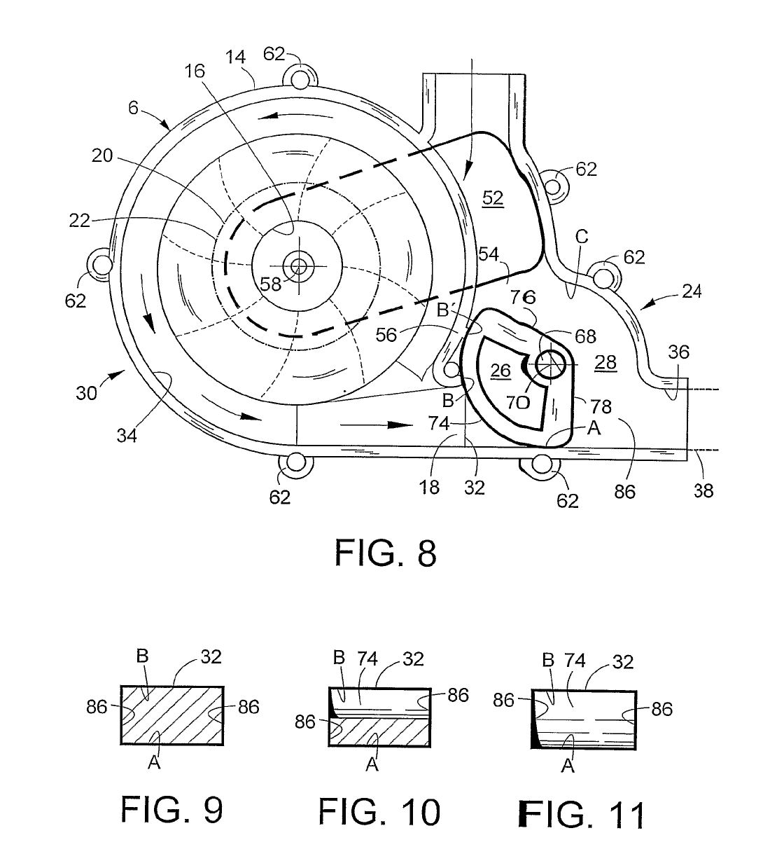

In FIG. 8, the valve body 26 is rotated still further in the counter clockwise direction, thus increasing the rotation angle β to approximately 90° in the shown embodiment. Now, the arced, first flow regulatory surface 74 of the valve body 26 is brought into abutment with the first valve body abutment surface A, thus blocking the inlet opening 32 to the valve chamber 28 completely in a fully closed position of the control valve 24, for example during an expiratory phase in the breathing pattern of the patient, eventhough a 100% closure of the control valve 24 is rare in the normal operation of the ventilator 1. It should be noted that, in the closed position of the control valve 24, the bypass opening 54 is instead fully open in order to allow air trapped in the hose 38, or outlet conduit to the facial mask 42, to feather back slightly as the patient expirates. However, due to the length and narrow cross-section of the outlet conduit 38, this air will not enter back into the gas flow generator chamber through the bypass conduit 52.

FIG. 9 shows a schematic view of the valve opening extent, as seen at the inlet opening 32 to the valve chamber 28, the control valve 24 being in its fully open position as shown in FIG. 3. The hatched lines indicate the open flow cross-sectional area. In FIG. 10, the valve body 26 has been rotated to the regulating position shown in FIG. 6, whereas FIG. 11 shows the valve body 26 in the fully closed position of the control valve 24. According to the invention, the rotation of the valve body 26 about the rotational axis 70 results in a linear change of flow cross-sectional area at the inlet opening 32 to the valve chamber 28.

It is to be understood that the invention is by no means limited to the embodiments described above, and may be varied freely within the scope of the appended claims.

### LIST OF REFERENCE NUMERALS AND SIGNS

1. Ventilator
2. Schematic illustration of a patients nose
4. External Housing
6. Gas Flow Generator
8. Gas Inlet Conduit
10. Particle Filter
12. External opening of the gas inlet conduit
14. Gas Flow Generator Chamber
16. Gas inlet opening in gas flow generator chamber
18. Gas outlet opening in gas flow generator chamber
20. Fan Rotor Wheel
22. Electric Motor
24. Control Valve
26. Valve Body
28. Valve Chamber
28a. Section of valve chamber
28b. Section of valve chamber (not shown)
30. Combined gas flow generator & control valve housing
30a. First shell
30b. Second shell (not shown)
32. Inlet Opening to Valve Chamber
34. Peripheral outer wall of gas flow generator chamber
36. Outlet Opening of Valve Chamber
38. Outlet Conduit
40. Air Humidifier
42. Patient interface means (facial mask)
44. Exhaust Openings
46. Flow Sensor
48. Other Optional Sensors
50. Control Unit
52. Bypass Conduit
54. Bypass Opening
56. Peripheral inner wall of bypass conduit
58. Rotational axis 58 of fan rotor wheel
60. Mounting Screws
62. Screw Lugs
64. Outline Periphery of the Shells
66. Electric Stepper Motor
68. Stepper motor Shaft
70. Rotational Axis of Valve Body
72. Trough hole in valve body for stepper motor shaft
74. Arced, first flow regulatory surface on valve body
76. Straight, second flow regulatory surface on valve body
78. Straight, third flow regulatory surface on valve body
80. Rounded transitional portions on valve body
82. Recesses in Valve Body
84. Flat End Surfaces
86. Flat Sidewalls of Valve Chamber
A. First valve body abutment surface
B. Second valve body abutment surface
B′. Supplemental second valve body abutment surface
C. Third valve body abutment surface
α. Sector angle of valve body
β. Rotation angle of valve body

1. A ventilator (1) for supplying breathable gas to the airway of a patient with a respiratory disorder, comprising:
a gas flow generator (6), such as an electric fan, for generating a flow of said breathable gas to the patient, said gas flow generator (6) comprising a gas flow generator chamber (14) provided with a gas inlet opening (16) and a gas outlet opening (18);
a control valve (24) for controlling the flow and/or pressure of the gas distributed to the patient, said control valve (24) comprising a valve body (26) which is rotatably arranged about a rotational axis (70) within a valve chamber (28), characterized in
that the rotational axis (70) of the valve body (26) is substantially perpendicular to the exhaust direction of the breathable gas at the gas outlet opening (18) of the gas flow generator (6);
that the valve body (26) essentially exhibits the shape of a sector of a circle in a plane perpendicular to said rotational axis (70), in such a way that an arced first flow regulatory surface (74) is formed along the circular arc of said sector, and that second (76) and third (78) essentially straight flow regulatory surfaces, respectively, are formed along the two diverging sides of said sector;
that said valve chamber (28) exhibits two mutually opposing, essentially flat sidewalls (86) both extending in a plane perpendicular to said rotational axis (70) of the valve body (26), and
that first, second and third valve body abutment surfaces (A, B, C), respectively, extend between said sidewalls (86) of the valve chamber (28), said valve body abutment surfaces (A, B, C) being arranged for abutting contact with the arced first flow regulatory surface (74) of the valve body (26), depending on the angular position of the valve body (26) within the valve chamber (28), wherein
said first valve body abutment surface (A) is located on one side of an inlet opening (32) to the valve chamber (28), said inlet opening (32) being connected to the gas outlet opening (18) of the gas flow generator chamber (14);
said second valve body abutment surface (B) is located between said inlet opening (32) and a bypass opening (54) arranged for directing a portion of the gas flow back into said gas flow generator (6) via a bypass conduit (52) connected to the gas inlet opening (16) of the gas flow generator chamber (14), and
said third valve body abutment surface (C) is located on an opposing side of said bypass opening (54) with respect to said second valve body abutment surface (B).

2. Ventilator (1) according to claim 1, characterized in that the valve body (26) exhibits rounded transitional portions (80) between the arced first flow regulatory surface (74) and the second (76) and a third (78) essentially straight flow regulatory surfaces.

3. Ventilator (1) according to claim 1, characterized in that the valve body (26) is formed in such a way that a sector angle (α) between the second (76) and third (78) flow regulatory surfaces is between 90°-160°.

4. Ventilator (1) according to claim 3 characterized in that said sector angle (α) is between 110°-130°.

5. Ventilator (1) according to claim 3 or 4, characterized in that said sector angle (α) is 120°.

6. Ventilator (1) according to any of the preceding claims, characterized in
said gas flow generator chamber (14) and said valve chamber (28) are integrally formed in a combined gas flow generator & control valve housing (30), and that
said valve chamber (28) is located in immediate conjunction to the gas outlet opening (18) of the gas flow generator chamber (14) within said combined gas flow generator & valve housing (30).

7. Ventilator (1) according to claim 6, characterized in that said gas outlet opening (18) of the gas flow generator chamber (14) also defines an inlet opening (32) to said valve chamber (28).

8. Ventilator (1) according to any of the preceding claims, characterized in that said rotational axis (70) of the valve body (26) is parallel to a rotational axis (58) of a fan rotor wheel (20) in said gas flow generator chamber (14).

9. Ventilator (1) according to any of claims 6 to 8, characterized in that an electric stepper motor is attached to the combined gas flow generator & control valve housing (30), said electric stepper motor (66) having a stepper motor shaft (68) coupled to the valve body (26) in said valve chamber (28).

10. Ventilator (1) according to claim 9 characterized in that the valve body (26) is provided with a through hole (72), said through hole (72) having a cross-sectional shape such that the valve body (26) is rotationally fixed relative to the stepper motor shaft (68), whilst being freely slidably arranged in an axial direction of said stepper motor shaft (68) for easy insertion or removal of the valve body (26) in the valve chamber (28).
